(19) United States
(12) Patent Application Publication
(10) Pub. No.: US 2006/0052751 A1
(43) Pub. Date: Mar. 9, 2006

(54) Fast perfusion cell chamber

(76) Inventors: Sepehr Eskandari, Diamond Bar, CA (US); Michael J. Errico, Tustin, CA (US)

Correspondence Address: Sepehr Eskandari, Ph.D.;Biological Sciences Dept., Cal. Poly University Pomona, 3801 West Temple Avenue, Pomona, CA, 91768-4032, US

(21) Appl. No.: 11/109,467

(22) Filed: Apr. 19, 2005

Related U.S. Application Data
(60) Provisional application No. 60/563,912, filed on Apr. 20, 2004.

(51) Int. Cl. A61M 5/178

(52) U.S. Cl. 604/164.11

(57) ABSTRACT

The present invention is a chamber used in recording electrophysiological responses of Xenopus laevis oocytes produced from rapid solution concentration changes. The chamber (8) contains a support base (6), attached at one end to the bottom of the chamber, and with support prongs at the other end (12). The cell (10; Xenopus laevis oocyte) rests on the support base. The current electrode (4) and voltage electrode (5) provide additional support for the cell (10). The solution flows downward in a vertical direction from a manifold unit (2) onto the cell. The supporting structures for the cell in the presence of minimal solution permit very high rates of solution changes while preserving the integrity of the cell membrane, such that fast electrophysiological signals potential may be measured and recorded accurately.

Pursuant to 35 U.S.C. §119(e), this patent application hereby references, incorporates by reference, and claims the benefit of U.S. Provisional Application No. 60/563,912, filed Apr. 20, 2004 and naming inventors Sepehr Eskandari and Michael J. Errico.

### BACKGROUND OF THE INVENTION

It is commonly known that, when using the two-microelectrode voltage-clamp technique in large cells such as Xenopus laevis oocytes (˜1 mm in diameter), rapid changes in cell membrane current may only be observed if the corresponding solution change is also very rapid. Indeed, the more nearly instantaneous the solution change is, the more readily membrane current changes can be resolved and measured, which might otherwise be missed or remain unobserved because they occur too rapidly. The present invention allows a rapid solution change around intact, voltage-clamped Xenopus laevis oocytes, while maintaining the integrity of the cell membrane during an experiment. This invention will facilitate the collection and recording of fast electrophysiological data obtained from Xenopus laevis oocytes expressing a variety of electrogenic membrane transport proteins.

Commonly-used cell chambers typically permit solution changes in no less than 30 seconds. Chambers designed specifically for rapid perfusion, accomplish solution changes in the order of ≧300 ms. The present invention permits solution changes on the order of 10-20 ms, with no substantial changes in cell membrane integrity. Most of the currently-used slower chambers achieve a solution change with a horizontal solution flow. Farb et al., (U.S. Pat. No. 6,048,722) describe such a system. While this design is adequate at slower flow rates, the key feature of the present invention is a vertical solution flow with adequate structural support for the cell, which minimizes cell movement while maximizing solution flow rate.

Fast transient kinetic methods provide invaluable information about the mechanism of protein function (Gutfreund, 1995). Several methods are utilized to perturb protein kinetics in order to gather information about the mechanism of function. Such perturbations use temperature, pressure, and concentration jumps, and for membrane proteins, transmembrane voltage jumps. Concentration jumps are difficult to achieve, and are especially troublesome for integral membrane proteins. The difficulty increases exponentially for membrane proteins in large cells (such as Xenopus oocytes) possessing microvilli. At least two factors lead to this difficulty: (i) in many cases, diffusion of the species of interest to the reaction region (i.e., mouth of channel, binding site of transporter or enzyme, etc.) is the rate limiting step in the overall reaction process. This challenge is compounded in cells possessing microvilli, where ligand diffusion in the space in between microvilli may become rate limiting; and (ii) in the case of membrane proteins, the unstirred layer becomes an important problem which is very difficult to overcome (Barry and Diamond, 1984).

A great deal has been learned about the rapid kinetics of electrogenic Na⁺-coupled cotransporters. This knowledge has come primarily from the use of transmembrane voltage jumps (Eskandari et al., 1997, 2000; Forster et al., 1997, 1998; Hazama et al., 1997; Li et al., 2000; Loo et al., 1993, 2000; Lu and Hilgemann, 1999b; Mackenzie et al., 1996a,b; Mager et al., 1993, 1996; Sacher et al., 2002; Wadiche et al., 1995; Whitlow et al., 2003). Very little is known regarding fast ligand binding events in these proteins (Cammack et al., 1994; Lu and Hilgemann, 1999a; Mager et al., 1996). The present invention would permit experiments which probe such events. The device described by Farb et al., (U.S. Pat. No. 6,048,722) would be incapable of probing such fast electrophysiological signals because it does not allow solution changes as fast as those obtained with the chamber described in this document.

Examination of the rapid kinetics of electrogenic Na⁺-coupled transporters is hampered by several factors: (i) These proteins have low turnover rates and, therefore, the cotransport mode of operation does not give rise to detectable microscopic currents. With turnover rates ranging from 1-100 s⁻¹, microscopic current measurements are not technically possible for these proteins. Therefore, the patch-clamp method, which has proven very useful for ion channels, is largely without use for measuring the unitary conductance of electrogenic Na⁺-coupled cotransporters. (ii) Even when expressed in model cells such as Xenopus laevis oocytes, where a large number of copies may be expressed at the cell plasma membrane, the macroscopic currents may not reach more than tens of nanoamperes. (iii) Although, the giant-patch method may be used for macroscopic measurements of Na⁺-coupled transporters (Eskandari et al., 1999; Lu and Hilgemann, 1999a,b; Lu et al., 1995), this method requires a very high level of expression for successful recording; a situation that is uncommon for most transporters expressed in Xenopus oocytes. The problems noted above will be avoided if macroscopic measurements are made in intact oocytes.

### SUMMARY OF THE INVENTION

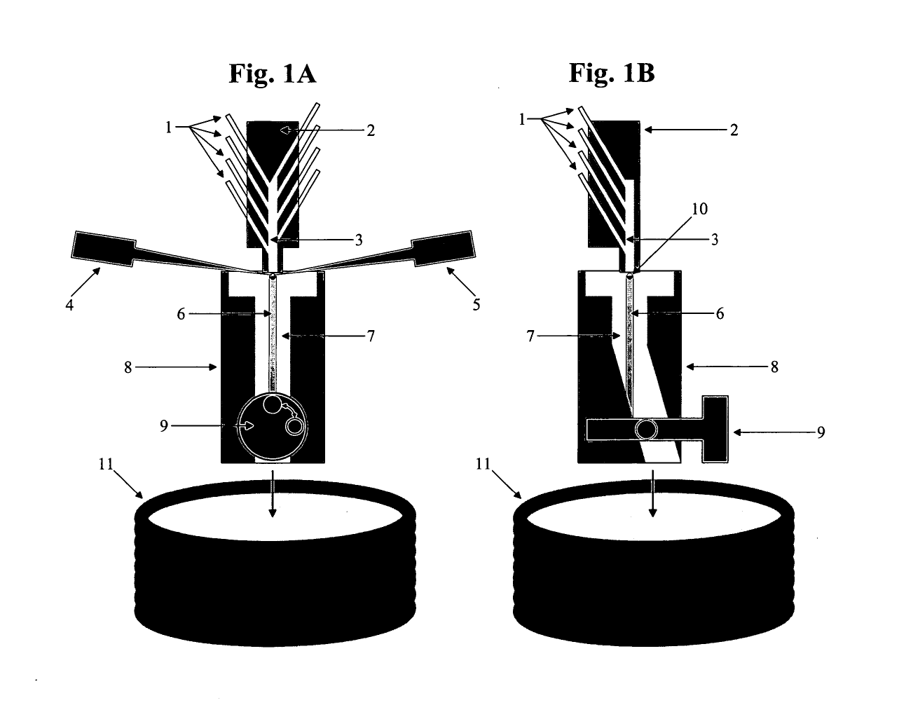

The usefulness of the Xenopus oocyte expression system for a variety of electrogenic membrane proteins has driven us to invent a novel rapid perfusion method which allows fast (≦20 ms) solution changes around intact, voltage-clamped Xenopus laevis oocytes (FIGS. 1-4). The present invention permits rapid solution changes to be made about voltage-clamped Xenopus laevis oocytes, while preserving the integrity of the cellular membrane. Defolliculated oocytes (intact vitelline membrane) and conventional two-electrode voltage clamp are used. For fast perfusion, the oocyte is placed in-line with the flow of buffer. The oocyte is supported primarily by an oocyte support base (6) equipped with oocyte support prongs (12), which prevent cellular movement. The current and voltage electrodes (4, 5) (for electrophysiological measurements) provide some support. Buffer flows from above the oocyte in a vertical direction (driven by gravity), at adjustable rates ranging from 1-50 ml/min. Healthy oocytes withstand maximal flow rates due to the chamber's support mechanisms.

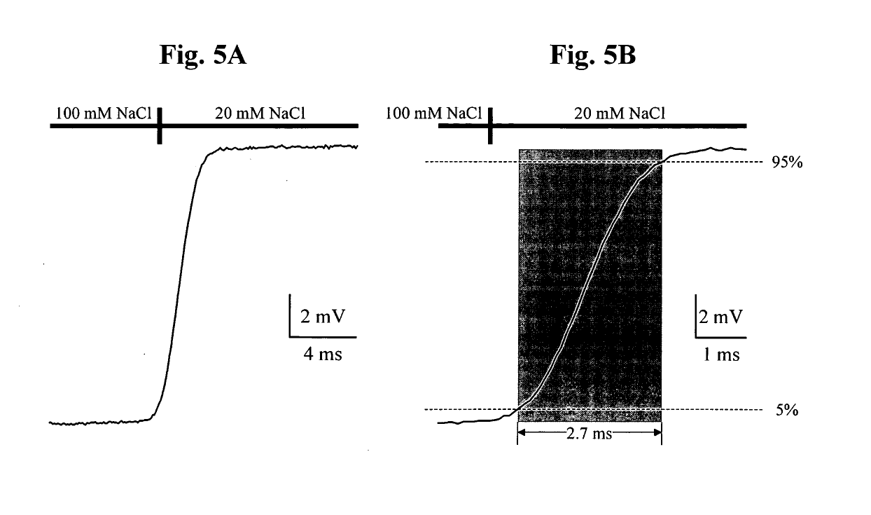

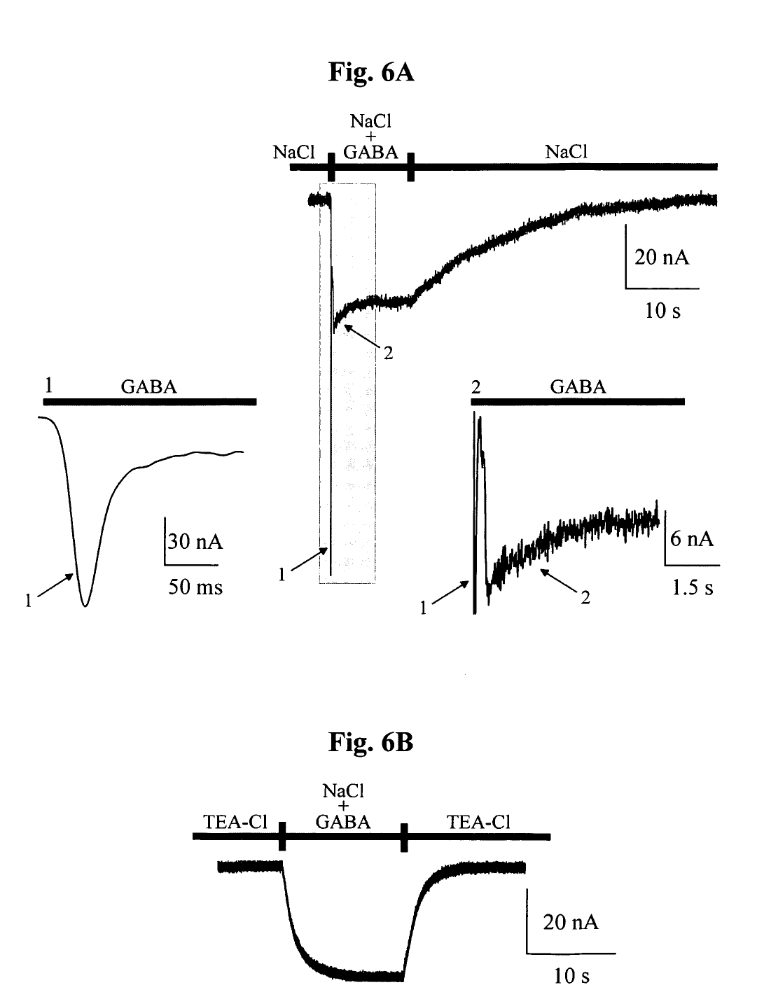

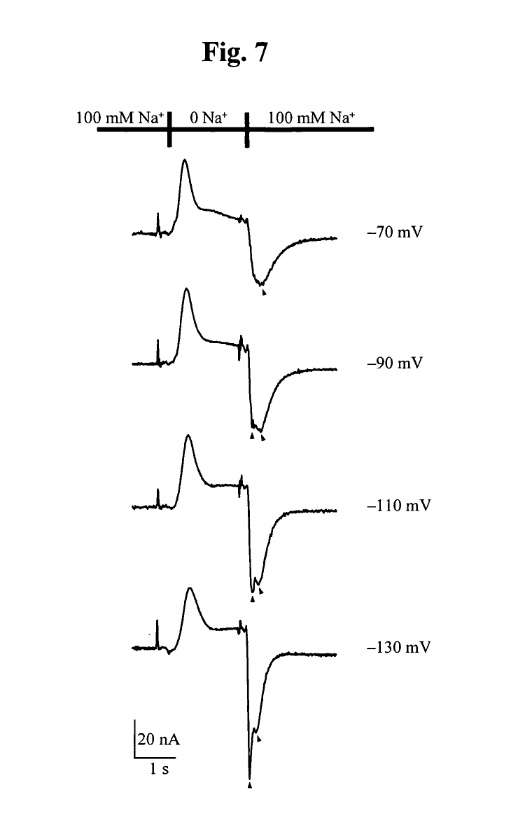

In the absence of oocyte in the chamber, the fluid exchange rate at the recording electrodes is ≈3 ms at maximal buffer flow rates (FIG. 5), the fastest solution change rate ever reported for Xenopus laevis oocytes. The exchange rate with oocyte in place is more difficult to determine due to the highly invaginated surface of the cell (i.e., microvilli), but is estimated to be no more than ≈20 ms. The geometry of the oocyte, microvilli in the oocyte plasma membrane, as well as the oocyte vitelline membrane most likely contribute to this slowing effect. Nevertheless, this speed of solution change is far superior to that of other systems designed specifically for intact oocytes (≧300 ms) (Madeja et al., 1991, 1995, 1997; Mager et al., 1996). A number of features of the system have been examined with oocytes expressing the human γ-aminobutyric acid transporter protein and compared with results obtained from traditional methods (FIGS. 6 and 7). This system permits examination of ligand interaction with electrogenic membrane proteins, a proposition which has heretofore been unattainable. The system also allows for rapid determination of kinetic parameters for drugs of interest.

In sum, the present invention is a rapid perfusion system which allows solution changes around intact, voltage-clamped oocytes. Solution change around the oocyte is complete in ≈20 ms, allowing inference of fast mechanistic information about ligand binding to Na⁺-coupled transporters. For the human Na⁺/Cl⁻/GABA transporter, this invention will permit numerous insightful observations to be made, which would otherwise remain unobservable. As the Xenopus laevis oocyte expression system is useful for examination of a variety of electrogenic membrane proteins, the present invention will allow a rich insight into the mechanism of function for a large number of membrane proteins, whose fast kinetics may not be studied by conventional methods.

### DRAWINGS—REFERENCE NUMERALS

1. Inlet lines of the manifold unit
2. Manifold unit
3. Main fluid line of the manifold unit
4. Current electrode
5. Voltage electrode
6. Oocyte support base
7. Fluid line of the chamber
8. Main perfusion chamber
9. Drain valve in the closed position
10. Xenopus laevis oocyte
11. Waste reservoir
12. Oocyte support prongs
13. Oocyte loading platform in the loading position
14. Oocyte loading platform in the retracted position
15. Drain valve in the open position

The following detailed description and annexed drawings are provided only for purposes of illustration of one possible embodiment of the present invention, and not for purposes of limitation of the appended claims.

### DRAWINGS—BRIEF DESCRIPTION

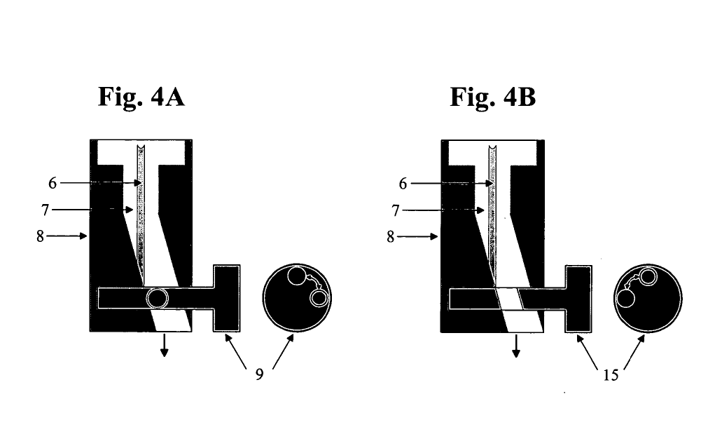

FIGS. 1A and 1B demonstrate the basic design of the perfusion chamber. Shown are the front and left views of the gravity-driven, vertical oocyte perfusion chamber. Software-controlled solenoid valves (not shown) control solution flow to the 10 input lines (1) of the manifold unit (2). The manifold unit (2) contains up to 10 inlet lines (1), which join to form the main fluid line of the manifold unit (2). Solution flows out of the manifold main fluid line (2), and onto a Xenopus laevis oocyte (10) positioned in the main perfusion chamber (8). The oocyte (10) rests on a base support (6) equipped with four support prongs (12), which snugly hold the oocyte in place, while minimizing surface contact with the oocyte plasma membrane. Minimization of surface contact with the oocyte plasma membrane while maintaining structural integrity at high solution flow rates are the bases for the improvements claimed in this invention. Beyond the oocyte, the solution enters the main fluid line (7) of the perfusion chamber (8). The solution exits the perfusion chamber (still under the force of gravity) through a drain valve (9; see also 15 in FIG. 4B), and finally enters a waste reservoir (11). For two-electrode voltage clamp, the oocyte (10) is impaled with the current (4) and voltage (5) electrodes. Thus, the oocyte is stabilized in an “oocyte pocket” by the base support (6), four supporting prongs (12), and two glass microelectrodes (4, 5).

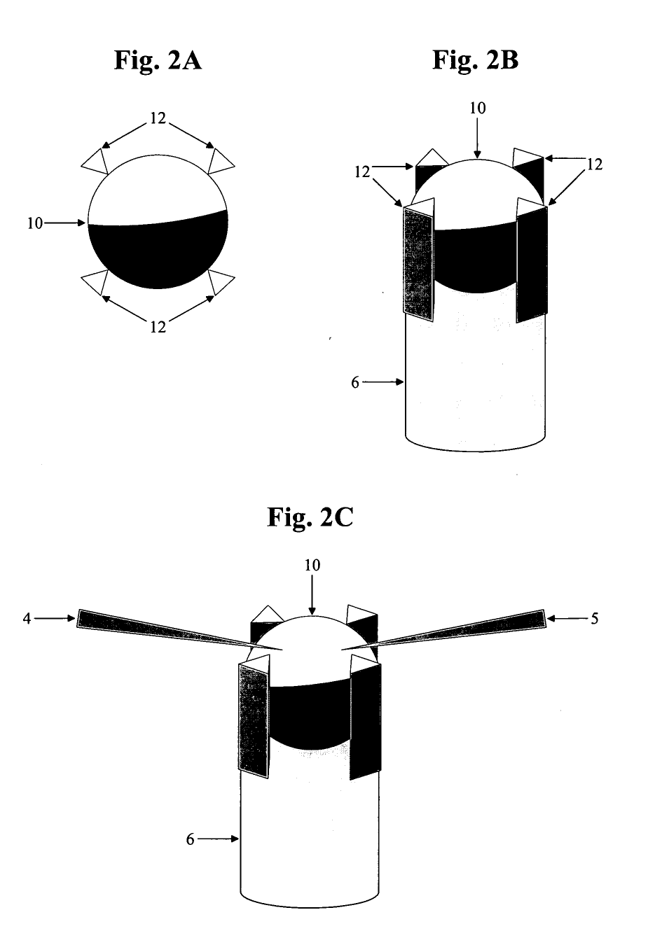

FIGS. 2A-2C show the main structural support elements for the oocyte (10). The oocyte support base (6) is equipped with four support prongs (12). This arrangement provides support for the oocyte while it minimizes surface contact with the oocyte plasma membrane. This is essential to rapid solution changes as it is desired that the perfusion solution nearly instantaneously bathes all regions of the cell surface. The current (4) and voltage (5) also enhance support by preventing lateral movements. In this “oocyte pocket” composed of the support base (6), support prongs (12), and current (4) and voltage (5) electrodes, the cell can withstand solution flow rates as high as 50 ml/min.

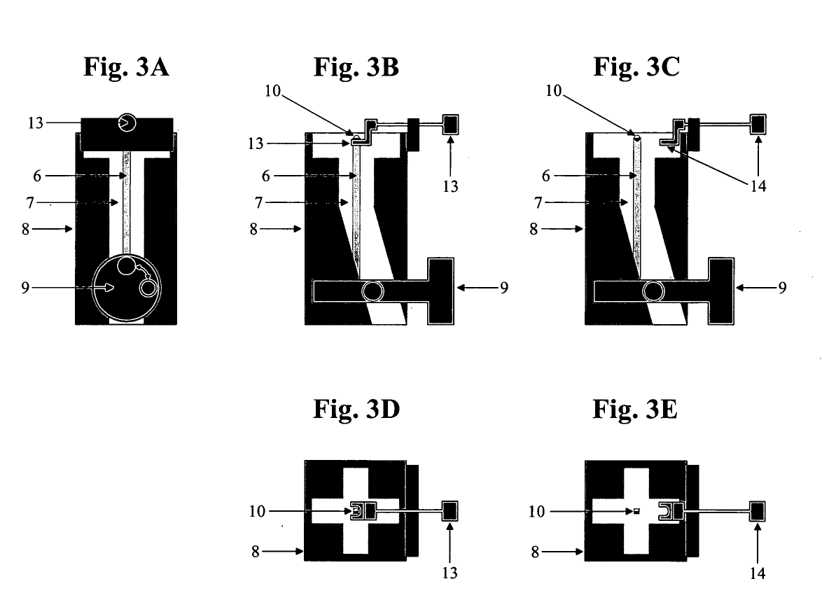

FIGS. 3A-3E show the mechanism used to place an oocyte (10) in the “oocyte pocket.” Because the oocyte support base (6) has a diameter (˜1.0 mm) that is approximately similar to that of the oocyte (˜1.0 mm), placing the oocyte into the “oocyte pocket” represents a challenge. To position the oocyte in the “oocyte pocket,” a loading platform (13, 14) is used. To mount the oocyte in the chamber, the loading platform (13) is moved around the oocyte support base (6) (see FIGS. 3B and 3D). After the oocyte is placed in the “oocyte pocket,” the loading platform (14) is retracted before oocyte impalement (see FIGS. 3C and 3E). Following oocyte impalement with the current and voltage electrodes, the manifold unit (2) is positioned directly above the oocyte (see FIG. 1).

FIGS. 4A and 4B show the drain mechanism for the perfusion chamber. Referring to FIG. 4A, because the cell needs to be bathed in a physiological saline solution at all times, throughout oocyte mounting (see FIG. 3) and impalement (see FIGS. 1 and 2), the drain valves remains closed (9), and the entire fluid line (7) of the perfusion chamber (8) is filled with physiological saline. Thus, oocyte mounting and impalement are carried out in the absence of solution flow (i.e., static solution). Referring to FIG. 4B, once gravity-driven perfusion begins (software-controlled opening of solenoid valves), constant flow through the chamber is ensured by opening the drain valve (15). Solution then exits the perfusion chamber and enters a waste reservoir (11).

FIGS. 5A and 5B show the speed of solution change in the proposed chamber. Referring to FIG. 5A, the time course of solution change in the chamber was examined in the absence of an oocyte. This recording was obtained by rapidly changing the perfusion solution (100 mM NaCl buffer) to a hypotonic solution (20 mM NaCl). The resulting junction potential at the recording electrode induces a current which follows the time course of the solution exchange in the chamber. Referring to FIG. 5B, fluid exchange at the recording electrodes is complete in ˜3 ms. The 5-95% rise time is 2.7 ms.

FIGS. 6A and 6B show a representative γ-aminobutyric acid (GABA) concentration jump in an oocyte expressing the GABA transporter isoform GAT4. The membrane potential (Vₘ) was held at −50 mV and the GABA concentration was 0.5 mM. The GABA-evoked inward current exhibited three distinct phases; (i) a rapid transient current (arrow at 1 and expanded in inset 1), (ii) a slow transient phase (arrow at 2 and expanded in inset 2), and (iii) a steady-state current. For the rapid transient phase, the time-to-peak was 20±5 ms, and its decay had a time constant of 15±2 ms (N=6). This rapid transient induced by a concentration jump represents the fastest ever reported with the two-electrode voltage clamp for any membrane protein expressed in the Xenopus laevis oocyte expression system. The slow transient phase exhibited mono-exponential decay to a steady-state with a time constant of 1.3±0.1 s (N=6). The steady-state current represents steady-state Na⁺/Cl⁻/GABA cotransport into the cell. Only the steady-state current is observed in conventional two-electrode voltage clamp records (not shown). The rapid transient represents fast GABA-evoked electrogenic transitions. Upon rapid removal of GABA, the GABA-evoked current exhibited mono-exponential decay back to baseline with a time constant of 4.0±0.6 s (N=6). Referring to FIG. 6B, if GABA and Na⁺ are simultaneously introduced, the rapid transient phase is absent and the evoked current follows a monoexponential behavior (τ=602±98 ms; N=6). The data suggest that Na⁺ binding to the transporter constitutes the rate-limiting step in the transport cycle. Because these events are very fast events, these measurements would not have been possible without the present invention. Thus, our perfusion chamber allows the acquisition of novel information from electrogenic membrane transport proteins expressed in oocytes.

FIG. 7 shows a representative Na⁺ concentration jump in an oocyte expressing the GABA transporter isoform GAT1 (Vₘ=−70 mV). Rapid Na⁺ removal from the bath evokes transient outward currents. This outward current may be interpreted as dissociation of positive charge (Na⁺) from the transporter. Upon rapid reintroduction of Na⁺ (100 mM) into the bath, a transient inward current is observed which is equal in magnitude to the transient outward component. Thus, Na⁺ removal and addition lead to capacitive charge movements out of and into the membrane electric field, respectively. At least two charge moving steps (early and late steps) are observed upon Na⁺ dissociation from and binding to the transporter. ON and OFF refer to Na⁺ binding to and dissociation from the transporter, respectively. The steady-state component is also seen in control cells, but the transients are not observed in control cells. Once again, the speed of solution change achieved has allowed these novel observations to be made for the GABA transporters. As mentioned above, these measurements are otherwise impossible in the intact oocyte.

### DETAILED DESCRIPTION—PREFERRED EMBODIMENT

As noted above, the following detailed description (and description of drawings above) are not meant to limit the instant claimed invention, inasmuch as alternate embodiments will be readily apparent to, and appreciated by those skilled in the art.

The preferred embodiment of the present invention is illustrated in FIGS. 1-4. The main chamber (8) is preferably made of moldable polyethylene plastic, and has approximate exterior measurements of 2.0 cm×2.0 cm×5.0 cm. The interior of the chamber (7) is preferably cylindrical, having a diameter of approximately 1.25 cm, and a length of approximately 4.5 cm. The oocyte support base (6) is fashioned from stainless steel rod (˜1 mm in diameter). One end is machined such that four support prongs (12) extend approximately 1 mm from the support base (FIGS. 2A-2C). The prongs typically have a length of ˜1 mm, and are spaced in such a way as to permit a single Xenopus oocyte to be placed in between them. Thus, the four prongs create a small and snug “oocyte pocket.” The exterior front side of the main chamber (8) contains a fitting to hold the oocyte loading platform (13) in place (FIGS. 3A-3E). For oocyte placement into the “oocyte pocket,” the loading platform (13) is positioned such that is makes physical contact with the oocyte support base (FIGS. 3B and 3D). After the oocyte is in position, the loading platform (14) is retracted (FIGS. 3C and 3E). The exterior sides of the chamber also contain fittings whereby agar bridges may be attached to retain electrical conductance. The current electrode (4) and voltage electrode (5) may be inserted into the oocyte while resting on the support base, either by entering the top of the chamber at an extreme angle, or preferably, as shown in FIG. 1A, by entering the chamber laterally through small notches on either side of the main chamber. The bottom of the chamber contains a drain valve (9, 15) with a small opening (3-6 mm) to allow solution to drain into a waste reservoir container (11). The drain valve is closed (9) when mounting and impaling oocytes, and is opened (15) during rapid perfusion (FIG. 4B).

Above the main perfusion chamber (8) is a manifold unit (2), which is also preferably made of moldable polyethylene plastic. The manifold unit is held in place by any kind of simple support stand. Ideally, the support stand is capable of being moved vertically, thereby permitting placement of the manifold unit at varying distances from the oocyte. The support stand should also move horizontally, so as to allow retraction for oocyte mounting and impalement, and forward movement, so as to allow positioning above the oocyte for rapid perfusion. A common solution flow tube can be fitted at the solution inflow end (1) of the manifold unit, and solution is thereby delivered through the manifold unit directly onto the resting oocyte. For optimal operation, the mouth of the manifold is held ˜1 cm above the oocyte (10) during fluid delivery. The solution flows rapidly past the oocyte (10) at adjustable rates ranging from 1-50 ml/min.

What is claimed is:

1. A fluid delivery apparatus comprising:
a chamber having a hollow shaft with interior walls, said chamber having an upper end and a lower end; and
a tubular-shaped support base placed within said hollow shaft of said chamber, said support base having an upper end and a lower end, said support base having a plurality of support prongs attached to the upper end of said support base, said support prongs being pointed upwardly such that a cell can be placed atop said support base and in-between said support prongs, said chamber having an opening positioned above said upper end of said support base, said chamber having an opening positioned below said upper end of said support base.

2. The fluid delivery apparatus according to claim 1, further comprising a manifold unit positioned above said upper end of said support base.

3. The fluid delivery apparatus according claim 2 wherein said manifold unit is capable of being moved toward and away from said upper end of said support base.

4. The fluid delivery apparatus according to claim 1 wherein said support base is affixed to an interior wall of said chamber.

5. The fluid delivery apparatus according to claim 1, wherein said tubular-shaped support base is not round.

6. The fluid delivery apparatus according to claim 1, further comprising a loading platform, said loading platform having an inside end and an outside end, said inside end of said loading platform having a groove capable of holding a cell, said outside end of said loading platform having a handle, said loading platform being slidingly engaged with the upper end of said chamber and capable of moving a cell such that the cell can be positioned atop said upper end of said support base.

7. The fluid delivery apparatus according to claim 2, further comprising a loading platform, said loading platform having an inside end and an outside end, said inside end of said loading platform having a groove capable of holding a cell, said outside end of said loading platform having a handle, said loading platform being slidingly engaged with the upper end of said chamber and capable of moving a cell such that the cell can be positioned atop said upper end of said support base.

8. The fluid delivery apparatus according to claim 1, further comprising a drain valve at said lower end of said chamber.

9. The fluid delivery apparatus according to claim 6, further comprising a drain valve at said lower end of said chamber.

10. A fluid delivery apparatus comprising:
a chamber having a hollow shaft with interior walls, said chamber having an upper end and a lower end; and
an elongated support base placed within said hollow shaft of said chamber, said support base having an upper surface forming a cupped-shape such that a cell can be placed atop said support base, said chamber having an opening positioned above said upper surface of said support base, said chamber having an opening positioned below said upper surface of said support base.

11. The fluid delivery apparatus according to claim 10, further comprising a manifold unit positioned above said upper surface of said support base.

12. The fluid delivery apparatus according to claim 11 wherein said manifold unit is capable of being moved toward and away from said upper surface of said support base.

13. The fluid delivery apparatus according to claim 10 wherein said support base is affixed to an interior wall of said chamber.

14. The fluid delivery apparatus according to claim 10, wherein said elongated support base is not round.

15. The fluid delivery apparatus according to claim 10, further comprising a loading platform, said loading platform having an inside end and an outside end, said inside end of said loading platform having a grooved surface, said loading platform being slidingly engaged with the upper end of said chamber and capable of moving a cell such that the cell can be positioned atop said upper surface of said support base.

16. The fluid delivery apparatus according to claim 11, further comprising a loading platform, said loading platform having an inside end and an outside end, said inside end of said loading platform having a grooved surface, said loading platform being slidingly engaged with the upper end of said chamber and capable of moving a cell such that the cell can be positioned atop said upper surface of said support base.

17. The fluid delivery apparatus according to claim 10, further comprising a drain valve at said lower end of said chamber.

18. The fluid delivery apparatus according to claim 15, further comprising a drain valve at said lower end of said chamber.
